(19) United States
(12) Patent Application Publication
(10) Pub. No.: US 2007/0178121 A1
(43) Pub. Date: Aug. 2, 2007

(54) METHODS FOR ENHANCING SKIN TREATMENTS

(75) Inventors: Eric R. First, Boston, MA (US); Ryan S. Irvine, Los Angeles, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 11/566,859

(22) Filed: Dec. 5, 2006

Related U.S. Application Data
(60) Provisional application No. 60/762,816, filed on Jan. 27, 2006.

(51) Int. Cl. A61K 39/08; A61K 8/73; A61K 8/64; A61K 31/728

(52) U.S. Cl. 424/239.1; 514/54; 424/70.13; 424/70.14

(57) ABSTRACT

The present invention relates to methods for enhancing the result of a skin treatment for a human patient. In some embodiments, the methods comprise the step of administering a neurotoxin to a skin area designated for the skin treatment. The skin treatment includes the application of a dermatologic agent and/or procedure.

### CROSS-REFERENCE TO RELATED APPLICATIONS

The present application claims the benefit of U.S. Application No. 60/762,816, filed Jan. 27, 2006, the content of which in its entirety is hereby incorporated by reference.

### BACKGROUND

The present invention relates to methods for improving a result of a skin treatment (e.g., application of a dermatologic/cosmetic agent or procedure) by enhancing the area of intended treatment with a neurotoxin, e.g. a botulinum toxin.

### Skin Overview

The skin is a complex membrane that performs many physiological functions such as metabolism, synthesis, temperature regulation, and excretion. Its upper layer, the stratum corneum, is considered to be the main barrier to the percutaneous penetration of exogenous materials. This barrier is also important in the maintenance of water within the body as well as in the absorption of pharmaceutical and other agents. As with other routes of delivery, transport of a medicament or application of a treatment or procedure across the skin is associated with several disadvantages; the main drawback being that not all are suitable candidates. A number of physicochemical parameters have been identified (such as molecular weight) that influence the diffusion process, and variations in permeation rates can occur between different skin models, patients, different races, and between young and old. The major challenge is overcoming the resistance of the skin to permeation in a reversible and non-damaging manner so as to achieve optimal outcomes of a medicament or procedure.

The dermis, called “true skin,” is the layer beneath the epidermis. Its major parts are collagen (a protein that adds strength), reticular fibers (thin protein fibers that add support), and elastic fibers (a protein that adds flexibility). The dermis has two layers: the papillary layer, which has loose connective tissue, and the reticular layer, which has dense connective tissue. These layers are so closely associated that they are difficult to differentiate.

The papillary layer lies directly beneath the epidermis and connects to it via papillae (finger-like projections). Some papillae contain capillaries that nourish the epidermis; others contain Meissner's corpuscles, sensory touch receptors. A double row of papillae in finger pads produces the ridged fingerprints on fingertips. Similar patterns in the ridged fingerprints on fingertips are on palms of the hands and soles of the feet. Fingerprints and footprints keep skin from tearing and aid in gripping objects.

The reticular layer of the dermis contains crisscrossing collagen fibers that form a strong elastic network. This network forms a pattern called cleavage (Langer's) lines. Surgical incisions that are made parallel to cleavage lines heal faster and with less scarring than those made perpendicular. Parallel incisions disrupt collagen fibers less and require less scar tissue (cells that aid in healing) to close up a wound.

The reticular layer also contains Pacinian corpuscles, sensory receptors for deep pressure. This layer contains sweat glands, lymph vessels, smooth muscle, and hair follicles, described in the discussion on hair follicles later in this overview.

The hypodermis (subcutaneous layer) lies beneath the dermis. Loose connective tissue such as adipose tissue (fat) insulates the body, conserving heat. It also contains blood vessels, lymph vessels, and the bases of hair follicles and sweat glands. The fat distribution in this layer gives the female form its characteristic curves.

### Sudoriferous (Sweat) and Sebaceous (Oil) Glands

Skin produces associated structures such as sudoriferous (sweat) glands and sebaceous (oil) glands. It also produces fingernails, hair, and sensory receptors that enable humans to feel pressure, temperature, and pain.

Both groups of sudoriferous glands (sweat glands) are in most of the body: eccrine glands are coiled ducts deep in the skin that connect to the surface; apocrine glands are in armpits, areolae of nipples, and the genital region. Eccrine glands secrete sweat, a mixture of 99 percent water and 1 percent salts and fats. In warm conditions with low humidity, perspiration (secretion of sweat) and evaporation cool the body Apocrine glands, which become active at puberty, are larger, deeper, and produce thicker secretions than eccrine glands. The apocrine glands secretions contain pheromones, substances that enable olfactory (sense of smell) communication with other members of the species. This communication provokes certain behavioral responses such as sexual arousal. Unlike eccrine glands that respond to heat, apocrine glands respond to stress and sexual activity by secreting sweat with a characteristic odor. This odor differs from body odor that results from bacteria decomposing skin secretions on the skin.

Ceruminous glands are modified apocrine glands in the external ear canal lining. They secrete cerumen (earwax), a sticky substance that is thought to repel foreign material.

Mammary glands in female breasts are modified apocrine glands. These glands are adapted to secrete milk instead of sweat.

Sebaceous glands (oil glands) are all over the body except on the palms of hands and soles of feet. The glands empty via ducts into the bases of hair follicles and secrete sebum (a mixture of fats, waxes, and hydrocarbons). Sebum keeps hair moist and prevents skin from drying. Sebaceous glands are numerous on the face and scalp. During puberty, increased sex hormone levels in the blood may produce excessive sebum. This over secretion plugs the gland and hair follicle, producing a skin disorder called acne.

### Hair and Nails

Hair is composed of cornified threads of cells that develop from the epidermis and cover most of the body. Each hair has a medulla, cortex, and cuticle. The medulla in the center contains soft keratin and air. The cortex, the innermost thickest layer, has the pigment that gives hair color. The cuticle, the outermost layer, has cells that overlap like scales. Both the cuticle and cortex have hard keratin.

The hair root in a hair follicle is embedded beneath the skin. The hair shaft protrudes from the skin. Hair sheds and is replaced constantly during growth and rest phases. Hair has a protective function: eyebrows keep sweat from running into the eyes, nose and ear hairs filter dust from the air, and scalp hairs protect against abrasion and overexposure to sun rays.

Hair follicles extend into the dermis; the deep ends expanded parts are called hair bulbs. A papilla (connective tissue protrusion that contains capillaries) protrudes into the hair bulb and provides nutrients for the growing hair. The hair follicle walls have an inner epithelial root sheath and an outer dermal root sheath. The epithelial root sheath has an inner and an outer layer that thins as it approaches the hair bulb. It becomes the matrix, the actively growing part of the hair bulb that produces the hair.

Erector pili muscles are smooth muscle cells attached to hair follicles. When they contract, they pull the hair into an upright position, causing skin dimples (goose bumps). The nervous system regulates these muscles; cold temperatures or fright can activate them.

Hair development begins in the third fetal month. By the fifth month, lanugo (thin hair) covers the fetus. At 5 months, lanugo disappears from every area except the scalp and eyebrows where coarser hair replaces it. Vellus (a film of delicate hair) eventually covers the rest of the body. Terminal hair is the early coarse scalp and eyebrow hair and later armpit and genital hair that grow during puberty. No new hair follicles develop after birth.

### Dermatologic and Cosmetic Treatments

With age, human faces begin to show the effects of gravity, sun exposure and years of facial muscle movement, such as smiling, chewing and squinting. The underlying tissues including the dermis, that keep skin looking youthful, begins to break down, often leaving laugh lines, smile lines, crow's feet or facial creases over the areas where this muscle movement occurs. Several treatments or agents exist that are utilized to enhance the dermis of the facial region or other regions of the body.

A number of non-surgical “refinishing” treatments are available to eliminate or soften imperfections on their facial skin and achieve a clearer, fresher look. These treatments include for example; glycolic acids (sometimes called “fruity acids”), which are natural fruit substances blended into facial preparations and are used to eliminate rough or dried surface skin, Retin-A®, a vitamin A-enriched cream that changes the cellular metabolism of the skin's surface and is used to combat fine facial wrinkles and blotches from sun damage. Soft-tissue fillers such as; injectable collagen or fat can fill in lines and creases.

When injected beneath the skin, fillers act to plump up creased and sunken areas of the face and also add fullness to the lips and cheeks. Injectable fillers may be used alone or in conjunction with a resurfacing procedure, such as a laser treatment, or a recontouring procedure, such as a facelift.

Additional examples of agents include, but are not limited to: corticosteroid, emollient, wrinkle modifiers, growth factor, moisturizer, peptide, antioxidant, keratolytic, retinoid, deoxyribonucleic acid, various acne agents, enzyme, ascorbic acid, anticellulite, growth factor, glycosaminoglycans, facial emollient, formulation thereof, and combinations thereof.

Examples of dermatologic or cosmetic treatments include, but are not limited to: varicose vein treatment, thermage treatment in facial rejuvenation procedures, filler-based treatment and procedures, high intensity focused ultrasound treatment, hormonal treatment for facial enhancement, laser treatment in hair removal procedures, laser (YAG) treatment in facial rejuvenation procedures, tattoo removal treatments hair removal treatment, microabrasion and dermabrasion therapies, hair loss treatment, DNA facial enhancement treatment, intense pulsed light photo rejuvenation procedures, anesthetic treatment prior to facial enhancement therapies, anticellulite treatment, facial enhancement, aesthetician treatments, massage treatment, physical and occupational therapy and chiropractic procedures or combinations thereof.

Each of these treatments can be used alone, or are frequently prescribed in conjunction with other aesthetic procedures, such as a dermabrasion or a chemical peel.

### Issues with Existing Treatments

Collagen is a naturally occurring protein that provides support to various parts of the human body: the skin, the joints, the bones and the ligaments. Injectable collagen, patented by the Collagen Corporation under the trade names Zyderm and Zyplast, is derived from purified bovine collagen. The purification process creates a product similar to human collagen. Injectable collagen received approval from the Food and Drug Administration in 1981. It is produced in various thicknesses to meet individual patient needs. Collagen is used primarily to fill wrinkles, lines and scars on the face and sometimes the neck, back and chest.

Treatment with collagen can begin after a skin test determines if the person is not allergic to the substance. The collagen is injected using a fine needle inserted at several points along the edge of the treatment site. If a local anesthesia has not been used, there may be some minor stinging or burning as the injections are administered. Injected material is eventually metabolized by the body.

Injectables such as collagen, are usually not sufficient for severe surface wrinkles on the face, such as multiple vertical “lipstick lines” that sometimes form around the mouth. Alternatives include a resurfacing technique, such as chemical peel, dermabrasion or laser treatments. Rather than filling in facial lines, resurfacing methods strip away the outer layers of the skin to produce a smoother appearance.

Resurfacing techniques such as dermabrasion and dermaplaning can smooth scars left by acne, accidents, or previous surgery, as well as fine facial wrinkles, especially those around the mouth. These procedures are normally safe. The most common risk in individuals with sensitive skin or underlying conditions such as; rosacea, is redness, pain and inflammation. Changes in skin pigmentation and permanent darkening of the skin, usually caused by exposure to the sun in the days or months following surgery, may occur in some individuals.

Other resurfacing and hair removal techniques including various types of laser therapy, a can cause significant redness, pain and inflammation in a person with sensitive skin. Patients with tight skin or deeper folds, may have less than an optimal response, requiring multiple visits, and often less than optimal outcomes.

The long term safety of dermatologic agents such as glycolic acids used in cosmetic procedures is not well known. Therefore, any reduction in the exposure time or amount of the agent would improve the safety and reduce unforeseen adverse events.

### Botulinum Toxin

The genus Clostridium has more than one hundred and twenty seven species, grouped according to their morphology and functions. The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of a commercially available botulinum toxin type A (purified neurotoxin complex)¹ is a LD₅₀ in mice (i.e. 1 unit). One unit of BOTOX® contains about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1976) (where the stated LD₅₀ of botulinum toxin type A of 0.3 ng equals 1 U is corrected for the fact that about 0.05 ng of BOTOX® equals 1 unit). One unit (U) of botulinum toxin is defined as the LD₅₀ upon intraperitoneal injection into female Swiss Webster mice weighing 18 to 20 grams each.

Seven, generally immunologically distinct botulinum neurotoxins have been characterized, these being respectively botulinum neurotoxin serotypes A, B, C₁, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is Available from Allergan, Inc., of Irvine, Calif. under the tradename BOTOX® in 100 unit vials) botulinum toxin type B. Additionally, botulinum toxin type B has been determined to be non-toxic in primates at a dose of 480 U/kg which is about 12 times the primate LD₅₀ for botulinum toxin type A. Moyer E et al., Botulinum Toxin Type B: Experimental and Clinical Experience, being chapter 6, pages 71-85 of “Therapy With Botulinum Toxin”, edited by Jankovic, J. et al. (1994), Marcel Dekker, Inc. Botulinum toxin apparently binds with high affinity to cholinergic motor neurons, is translocated into the neuron and blocks the release of acetylcholine. Additional uptake can take place through low affinity receptors, as well as by phagocytosis and pinocytosis.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C₁ was originally thought to cleave only syntaxin, but was found to cleave syntaxin and SNAP-25. Each of the botulinum toxins specifically cleaves a different bond, except botulinum toxin type B (and tetanus toxin) which cleave the same bond. Each of these cleavages block the process of vesicle-membrane docking, thereby preventing exocytosis of vesicle content.

Botulinum toxins have been used in clinical settings for the treatment of neuromuscular disorders characterized by hyperactive skeletal muscles (i.e. motor disorders). In 1989 a botulinum toxin type A (Allergan, Inc., BOTOX®) complex was approved by the U.S. Food and Drug Administration for the treatment of blepharospasm, strabismus and VII nerve related disorder. Subsequently, in 2000, both a botulinum toxin type B (Elan, Inc., MYOBLOC™) and a botulinum toxin type A (Allergan, Inc., BOTOX®), were approved by the FDA for the treatment of cervical dystonia. Furthermore, a botulinum toxin type A (Allergan, Inc., BOTOX®), was FDA-approved for the treatment of glabellar lines in 2002 and for severe primary axillary hyperhidrosis in 2004. Non-type A botulinum toxin serotypes apparently have a lower potency and/or a shorter duration of activity as compared to botulinum toxin type A. Clinical effects of peripheral intramuscular botulinum toxin type A are usually seen within one week of injection. The typical duration of symptomatic relief from a single intramuscular injection of botulinum toxin type A averages about three months, although significantly longer periods of therapeutic activity have been reported.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell types. See e.g. Biochem J 1; 339 (pt 1):159-65:1999, and Mov Disord, 10(3):376:1995 (pancreatic islet B cells contains at least SNAP-25 and synaptobrevin).

The molecular weight of the botulinum neurotoxin component protein molecule, for all seven of the known botulinum toxin serotypes, is about 150 kD. Interestingly, the botulinum toxins are released by Clostridial bacterium as complexes, comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C₁ are apparently produced as only a 700 kD or 500 kD complexes. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain various compositions of non-toxin hemaglutinin proteins and non-toxin and non-toxic nonhemaglutinin proteins; thus the variance in weights of the entire complexes. These non-toxin proteins (which along with the botulinum toxin molecule comprise the relevant neurotoxin complex) may act to provide stability against denaturation to the botulinum toxin molecule and protection against digestive acids when toxin is ingested (Sharma Sahshi K, Singh B R. Enhancement of the endopepdidase activity of purified botulinum neurotoxins A and E by an isolated component of the native neurotoxin associated proteins. Biochemistry. pp 4791-4798: 2004. Additionally, it is possible that the larger (greater than about 150 kD molecular weight) botulinum toxin complexes may result in a slower rate of diffusion of the botulinum toxin away from a site of intramuscular injection of a botulinum toxin complex (Schantz E J, Johnson E A (1992) Properties and use of botulinum toxin and other microbial neurotoxins in medicine. Microbiol Rev_{—}56_{—}:80-99).

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Neurotoxins Inhibit Noradrenaline Release From Cultured Mouse Brain, J Neurochem 51(2); 522-527:1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165; 675-681:1987. Botulinum toxin type A has also been shown to inhibit mediators (substance P, cGRP, and glutamate involved in pain and inflammation (Cui M, Aoki K R, Mechanisms of the antinociceptive effect of subcutaneous BOTOX): Inhibition of peripheral and central nonciceptive processing. Pain. 158-162; 2004; Durham P L, Cady R, Blumenfled A J. Regulation of calcitonin gene-related peptide secretion from trigeminal nerve cells by botulinum toxin type A: Implications for migraine therapy. Headache. pp 35-43; 2004.

Thus, when adequate concentrations are used, stimulus-evoked release of most neurotransmitters is blocked by botulinum toxin. See e.g. Pearce, L. B., Pharmacologic Characterization of Botulinum Toxin For Basic Science and Medicine, Toxicon 35(9); 1373-1412 at 1393; Bigalke H., et al., Botulinum A Neurotoxin Inhibits Non-Cholinergic Synaptic Transmission in Mouse Spinal Cord Neurons in Culture, Brain Research 360; 318-324:1985; Habermann E., Inhibition by Tetanus and Botulinum A Toxin of the release of [³H]Noradrenaline and [³H]GABA From Rat Brain Homogenate, Experientia 44; 224-226:1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316; 244-251:1981, and; Jankovic J. et al., Therapy With Botulinum Toxin, Marcel Dekker, Inc., (1994), page 5.

Botulinum toxin type A can be obtained by establishing and growing cultures of Clostridium botulinum in a fermentor and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of ≧3×10⁷ U/mg, an A₂₆₀/A₂₇₈ of less than 0.60 and a distinct pattern of banding on gel electrophoresis. The known Shantz process can be used to obtain crystalline botulinum toxin type A, as set forth in Shantz, E. J., et al, Properties and use of Botulinum toxin and Other Microbial Neurotoxins in Medicine, Microbiol Rev. 56; 80-99:1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

Botulinum toxins and/or botulinum toxin complexes can be obtained from List Biological Laboratories, Inc., Campbell, Calif.; the Centre for Applied Microbiology and Research, Porton Down, U.K.; Wako (Osaka, Japan), Metabiologics (Madison, Wis.) as well as from Sigma Chemicals of St Louis, Mo. Pure botulinum toxin can also be used to prepare a pharmaceutical composition.

As with enzymes generally, the biological activities of the botulinum toxins (which are intracellular peptidases) is dependant, at least in part, upon their three dimensional conformation. Thus, botulinum toxin type A is detoxified by heat, various chemicals surface stretching and surface drying. Additionally, it is known that dilution of the toxin complex obtained by the known culturing, fermentation and purification to the much lower toxin concentrations used for pharmaceutical composition formulation, results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin can stabilized with a stabilizing agent such as albumin and gelatin.

A commercially available botulinum toxin containing pharmaceutical composition is sold under the trademark BOTOX® (available from Allergan, Inc., of Irvine, Calif.). BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks. Neurology, 48:249-53:1997.

It has been reported that botulinum toxin type A has been used in clinical settings as follows:

(1) about 75-125 units of BOTOX® per intramuscular injection (multiple muscles) to treat cervical dystonia;
(2) 5-10 units of BOTOX® per intramuscular injection to treat glabellar lines (brow furrows) (5 units injected intramuscularly into the procerus muscle and 10 units injected intramuscularly into each corrugator supercilii muscle);
(3) about 30-80 units of BOTOX® to treat constipation by intrasphincter injection of the puborectalis muscle;
(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.
(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).
(6) to treat upper limb spasticity following stroke by intramuscular injections of BOTOX® into five different upper limb flexor muscles, as follows:
(a) flexor digitorum profundus: 7.5 U to 30 U
(b) flexor digitorum sublimus: 7.5 U to 30 U
(c) flexor carpi ulnaris: 10 U to 40 U
(d) flexor carpi radialis: 15 U to 60 U
(e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.
(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

Additionally, intramuscular botulinum toxin has been used in the treatment of tremor in patients with Parkinson's disease, although it has been reported that results have not been impressive. Marjama-Jyons, J., et al., Tremor-Predominant Parkinson's Disease, Drugs & Aging 16(4); 273-278:2000.

It is known that botulinum toxin type A can have an efficacy for up to 12 months (European J. Neurology 6 (Supp 4): S111-S1150:1999), and in some circumstances for as long as 27 months, when used to treat glands, such as in the treatment of hyperhydrosis. See e.g. Bushara K., Botulinum toxin and rhinorrhea, Otolaryngol Head Neck Surg 1996; 14(3):507, and The Laryngoscope 109:1344-1346:1999. However, the usual duration of an intramuscular injection of Botox® is typically about 3 to 4 months.

The success of botulinum toxin type A to treat a variety of clinical conditions has led to interest in other botulinum toxin serotypes. There are three commercially available botulinum type A preparations for use in humans: BOTOX®, available from Allergan, Inc., of Irvine, Calif.; DYSPORT®, available from Beaufour Ipsen, Porton Down, England; and Xeomin®, available from Merz Pharmaceuticals GmbH, Frankfurt, Germany. A Botulinum toxin type B preparation (MYOBLOC®) is available from Elan Pharmaceuticals of San Francisco, Calif.

In addition to having pharmacologic actions at the peripheral location, botulinum toxins may also have inhibitory effects in the central nervous system. Work by Weigand et al, Nauny-Schmiedeberg's Arch. Pharmacol. 1976; 292, 161-165, and Habermann, Nauny-Schmiedeberg's Arch. Pharmacol. 1974; 281, 47-56 showed that botulinum toxin is able to ascend to the spinal area by retrograde transport. As such, a botulinum toxin injected at a peripheral location, for example intramuscularly, may be retrograde transported to the spinal cord.

U.S. Pat. No. 5,989,545 discloses that a modified clostridial neurotoxin or fragment thereof, preferably a botulinum toxin, chemically conjugated or recombinantly fused to a particular targeting moiety can be used to treat pain by administration of the agent to the spinal cord.

It has been reported that use of a botulinum toxin to treat various spasmodic muscle conditions can result in reduced depression and anxiety, as the muscle spasm is reduced. Murry T., et al., Spasmodic dysphonia; emotional status and botulinum toxin treatment, Arch Otolaryngol 1994 March; 120(3): 310-316; Jahanshahi M., et al., Psychological functioning before and after treatment of torticollis with botulinum toxin, J Neurol Neurosurg Psychiatry 1992; 55(3): 229-231. Additionally, German patent application DE 101 50 415 A1 discusses intramuscular injection of a botulinum toxin to treat depression and related affective disorders.

A botulinum toxin has also been proposed for or has been used to treat skin wounds (U.S. Pat. No. 6,447,787), various autonomic nerve dysfunctions (U.S. Pat. No. 5,766,605), tension headache, (U.S. Pat. No. 6,458,365), migraine headache pain (U.S. Pat. No. 5,714,468), sinus headache (U.S. patent application Ser. No. 429,069), post-operative pain and visceral pain (U.S. Pat. No. 6,464,986), neuralgia pain (U.S. patent application Ser. No. 630,587), hair growth and hair retention (U.S. Pat. No. 6,299,893), dental related ailments (U.S. provisional patent application Ser. No. 60/418,789), fibromyalgia (U.S. Pat. No. 6,623,742), various skin disorders (U.S. patent application Ser. No. 10/731,973), motion sickness (U.S. patent application Ser. No. 752,869), psoriasis and dermatitis (U.S. Pat. No. 5,670,484), injured muscles (U.S. Pat. No. 6,423,319) various cancers (U.S. Pat. No. 6,139,845), smooth muscle disorders (U.S. Pat. No. 5,437,291), down turned mouth corners (U.S. Pat. No. 6,358,917), nerve entrapment syndromes (U.S. patent application 2003 0224019), various impulse disorders (U.S. patent application Ser. No. 423,380), acne (WO 03/011333) and neurogenic inflammation (U.S. Pat. No. 6,063,768). Controlled release toxin implants are known (see e.g. U.S. Pat. Nos. 6,306,423 and 6,312,708) as is transdermal botulinum toxin administration (U.S. patent application Ser. No. 10/194,805).

Botulinum toxin type A has been used to treat epilepsia partialis continua, a type of focal motor epilepsy. Bhattacharya K., et al., Novel uses of botulinum toxin type A: two case reports, Mov Disord 2000; 15(Suppl 2):51-52.

It is known that a botulinum toxin can be used to: weaken the chewing or biting muscle of the mouth so that self inflicted wounds and resulting ulcers can heal (Payne M., et al, Botulinum toxin as a novel treatment for self mutilation in Lesch-Nyhan syndrome, Ann Neurol 2002 September; 52(3 Supp 1):S157); permit healing of benign cystic lesions or tumors (Blugerman G., et al., Multiple eccrine hidrocystomas: A new therapeutic option with botulinum toxin, Dermatol Surg 2003 May; 29(5):557-9); treat anal fissure (Jost W., Ten years' experience with botulinum toxin in anal fissure, Int J Colorectal Dis 2002 September; 17(5):298-302, and; treat certain types of atopic dermatitis (Heckmann M., et al., Botulinum toxin type A injection in the treatment of lichen simplex: An open pilot study, J Am Acad Dermatol 2002 April; 46(4):617-9).

Additionally, a botulinum toxin may have an effect to reduce induced inflammatory pain in a rat formalin model. Aoki K., et al, Mechanisms of the antinociceptive effect of subcutaneous Botox: Inhibition of peripheral and central nociceptive processing, Cephalalgia 2003 September; 23(7):649. Furthermore, it has been reported that botulinum toxin nerve blockage can cause a reduction of epidermal thickness. Li Y, et al., Sensory and motor denervation influences epidermal thickness in rat foot glabrous skin, Exp Neurol 1997; 147:452-462 (see page 459). Finally, it is known to administer a botulinum toxin to the foot to treat excessive foot sweating (Katsambas A., et al., Cutaneous diseases of the foot: Unapproved treatments, Clin Dermatol 2002 November-December; 20(6):689-699; Sevim, S., et al., Botulinum toxin-A therapy for palmar and plantar hyperhidrosis, Acta Neurol Belg 2002 December; 102(4):167-70), spastic toes (Suputtitada, A., Local botulinum toxin type A injections in the treatment of spastic toes, Am J Phys Med Rehabil 2002 October; 81(10):770-5), idiopathic toe walking (Tacks, L., et al., Idiopathic toe walking: Treatment with botulinum toxin A injection, Dev Med Child Neurol 2002; 44(Suppl 91):6), and foot dystonia (Rogers J., et al., Injections of botulinum toxin A in foot dystonia, Neurology 1993 April; 43(4 Suppl 2)).

Tetanus toxin, as wells as derivatives (i.e. with a non-native targeting moiety), fragments, hybrids and chimeras thereof can also have therapeutic utility. The tetanus toxin bears many similarities to the botulinum toxins. Thus, both the tetanus toxin and the botulinum toxins are polypeptides made by closely related species of Clostridium (Clostridium tetani and Clostridium botulinum, respectively). Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for ganglioside receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Contrarily, receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. Binz T. et al., The Complete Sequence of Botulinum Neurotoxin Type A and Comparison with Other Clostridial Neurotoxins, J Biological Chemistry 265(16); 9153-9158:1990.

Acetylcholine

Typically only a single type of small molecule neurotransmitter is released by each type of neuron in the mammalian nervous system, although there is evidence which suggests that several neuromodulators can be released by the same neuron. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the bag 1 fibers of the muscle spindle fiber, by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephine. In most instances acetylcholine has an excitatory effect. However, acetylcholine is known to have inhibitory effects at some of the peripheral parasympathetic nerve endings, such as inhibition of heart rate by the vagal nerve.

The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since, the preganglionic neurons of both the sympathetic and parasympathetic nervous system are cholinergic, application of acetylcholine to the ganglia will excite both sympathetic and parasympathetic postganglionic neurons.

Acetylcholine activates two types of receptors, muscarinic and nicotinic receptors. The muscarinic receptors are found in all effector cells stimulated by the postganglionic, neurons of the parasympathetic nervous system as well as in those stimulated by the postganglionic cholinergic neurons of the sympathetic nervous system. The nicotinic receptors are found in the adrenal medulla, as well as within the autonomic ganglia, that is on the cell surface of the postganglionic neuron at the synapse between the preganglionic and postganglionic neurons of both the sympathetic and parasympathetic systems. Nicotinic receptors are also found in many nonautonomic nerve endings, for example in the membranes of skeletal muscle fibers at the neuromuscular junction.

Acetylcholine is released from cholinergic neurons when small, clear, intracellular vesicles fuse with the presynaptic neuronal cell membrane. A wide variety of non-neuronal secretory cells, such as, adrenal medulla (as well as the PC12 cell line) and pancreatic islet cells release catecholamines and parathyroid hormone, respectively, from large dense-core vesicles. The PC 2 cell line is a clone of rat pheochromocytoma cells extensively used as a tissue culture model for studies of sympathoadrenal development. Botulinum toxin inhibits the release of both types of compounds from both types of cells in vitro, permeabilized (as by electroporation) or by direct injection of the toxin into the denervated cell. Botulinum toxin is also known to block release of the neurotransmitter glutamate from cortical synaptosomes cell cultures.

A neuromuscular junction is formed in skeletal muscle by the proximity of axons to muscle cells. A signal transmitted through the nervous system results in an action potential at the terminal axon, with activation of ion channels and resulting release of the neurotransmitter acetylcholine from intraneuronal synaptic vesicles, for example at the motor endplate of the neuromuscular junction. The acetylcholine crosses the extracellular space to bind with acetylcholine receptor proteins on the surface of the muscle end plate. Once sufficient binding has occurred, an action potential of the muscle cell causes specific membrane ion channel changes, resulting in muscle cell contraction. The acetylcholine is then released from the muscle cells and metabolized by cholinesterases in the extracellular space. The metabolites are recycled back into the terminal axon for reprocessing into further acetylcholine.

What is therefore needed is a method to enhance the effects of the dermatologic agent or procedure applied, reduce the amount of dermatologic agent applied and/or reduce the amount of time of the procedure.

### SUMMARY

The present invention meets this need, and provides methods for enhancing the effects of the dermatologic agent or procedure applied, reducing the amount of dermatologic agent applied and/or reducing the amount of time of the skin treatment procedure. In some embodiments, the method for enhancing a skin treatment comprises the step of administering a neurotoxin to a skin area designated for skin treatment. The dose of the neurotoxin administered can be calculated by determining a surface area (centimeter square) of the designated skin area, and multiplying the surface area by a unit of neurotoxin that is equivalent to about 1-5 units of botulinum toxin type A. In some embodiments, the enhancement of the skin treatment is the prolonging of the treatment result.

In some embodiments, the skin treatment comprises administering a dermatologic agent, e.g., an agent comprising a corticosteroids emollient, wrinkle modifiers, growth factor, moisturizer, peptide, antioxidant, keratolytic agent, retinoid, deoxyribonucleic acid, acne agents, enzymes, ascorbic acid, anticellulite, glycosaminoglycans, hyaluronic acid, crossed linked hyaluronic acid or combination thereof. In some embodiments, the skin treatment comprises administering a skin filler.

In some embodiments, the skin treatment comprises applying a dermatologic procedure, e.g., a procedure comprising a varicose vein treatment, thermage treatment in facial rejuvenation procedures, filler-based treatment, high intensity focused ultrasound treatment, hormonal treatment for facial enhancement, laser treatment for hair removal, laser treatment for facial rejuvenation, tattoo removal treatments, hair removal treatment, microabrasion and dermabrasion therapies, hair loss treatment, DNA facial enhancement treatment, intense pulsed light photo rejuvenation procedures, anesthetic treatment prior to facial enhancement therapies, anticellulite treatment, facial enhancement, aesthetician treatments, massage treatment, physical and occupational therapy, chiropractic procedures, or combination thereof.

In some embodiments, the method for enhancing a skin treatment comprises the step of administering a neurotoxin toxin to a skin area designated for skin treatment. In some embodiments, the skin treatment comprises administering a dermatologic agent comprising a corticosteroids emollient, wrinkle modifiers, growth factor, moisturizer, peptide, antioxidant, keratolytic agent, retinoid, deoxyribonucleic acid, acne agents, enzymes, ascorbic acid, anticellulite, or combination thereof. In some embodiments, the skin treatment excludes the treatment with a hyaluronic acid and/or cross linked hyaluronic acid. In some embodiments, the skin treatment comprises a varicose vein treatment, thermage treatment in facial rejuvenation procedures, filler-based treatment, high intensity focused ultrasound treatment, hormonal treatment for facial enhancement, laser treatment for hair removal, laser treatment for facial rejuvenation, tattoo removal treatments, hair removal treatment, microabrasion and dermabrasion therapies, hair loss treatment, DNA facial enhancement treatment, intense pulsed light photo rejuvenation procedures, anesthetic treatment prior to facial enhancement therapies, anticellulite treatment, facial enhancement, aesthetician treatments, massage treatment, physical and occupational therapy, chiropractic procedures, or combination thereof.

The present invention also provides a method for reducing an amount of a dermatologic agent used during treatment of a skin condition. In some embodiments, the present invention provides a method for reducing an amount of exposure time to a dermatologic procedure during treatment of a skin condition. In some embodiments, these methods comprise the step of administering a neurotoxin to a skin area designated for skin treatment, thereby reducing the amount of dermatologic agent used to obtain a treatment result at the designated skin area and/or reducing the amount of exposure time to a dermatologic procedure during treatment of a skin condition. For example, a higher amount of an identical dermatologic agent or longer exposure time to a dermatologic procedure is required to obtain the same treatment result if the botulinum toxin is not administered to the designated skin area.

In some embodiments, the present invention also provides for a method for treating a skin condition comprising the steps of (a) administering a neurotoxin to a skin area designated for skin treatment, (b) determining a muscle tone at the designated skin area, and (c) subjecting the designated skin area to a dermatologic agent or procedure. The step (b) of determining the muscle tone at the designated skin area allows for an appropriate reduction in the amount of dermatologic agent to be used and/or reduction in the amount of time exposure to a dermatologic procedure.

### DEFINITIONS

The term “neurotoxin” employed herein refers to one or more of a toxin made by a bacterium, for example, a Clostridium botulinum, Clostridium butyricum, Clostridium beratti, Clostridium tetani. In some embodiments, the neurotoxin is a botulinum toxin. The botulinum toxin may be a botulinum toxin type A, type B, type C₁, type D, type E, type F, or type G. In some embodiments, the neurotoxin is a botulinum toxin type A. Unless stated otherwise, the dose of the neurotoxin referenced herein is equivalent to that of a botulinum toxin type A. The assays required to determine equivalency to the therapeutic effectiveness of botulinum toxin type A at a certain dosage are well established.

Further, the following definitions apply herein:

“About” means approximately or nearly and in the context of a numerical value or range set forth herein means±10% of the numerical value or range recited or claimed.

“Alleviating” means a reduction in the occurrence of a pain, of a headache or of a symptom. Thus, alleviating includes some reduction, significant reduction, near total reduction, and total reduction. An alleviating effect may not appear clinically for between 1 to 7 days after administration of a Clostridial toxin to a patient.

“Botulinum toxin” means a botulinum neurotoxin as either pure toxin or complex, and excludes botulinum toxins which are not neurotoxins such as the cytotoxic botulinum toxins C₂ and C₃.

“Local administration” means administration (i.e. by a subcutaneous, intramuscular, subdermal or transdermal route) of a pharmaceutical agent to or to the vicinity muscle or a subdermal location by a non-systemic route. Thus, local administration excludes systemic (i.e. to the blood circulation system) routes of administration, such as intravenous or oral administration. Peripheral administration means administration to the periphery (i.e. to a location on or within a face, limb, trunk or head of a patient) as opposed to a visceral or gut (i.e. to the viscera) administration.

### DESCRIPTION

The present invention is based, in part, upon the discovery that an administration of a neurotoxin in conjunction with a skin treatment can enhance a treatment result of the treatment, reduce the amount of dermatologic agent required to obtain a treatment result and/or reduce the exposure time to a dermatologic procedure to obtain a treatment result. The neurotoxin can be administered before, at the same time, and/or after the skin treatment.

It has been demonstrated that denervation of the skin can cause the epidermis to began to degenerate or to become thinner. Hsieh S., et al., Modulation of keratinocyte proliferation by skin innervation, J Invest Dermatol. 1999 October; 113(4):579-86; Hsieh S., et al., Epidermal denervation and its effects on keratinocytes and Langerhans cells, J. Neurocytol. 1996 September; 25(9):513-24.); Chiang, et al., Regional difference in epidermal thinning after skin denervation, Exp Neurol 1998 November; 154(1):137-45; Li Y., et al., Sensory and motor denervation influence epidermal thickness in rat foot glabrous skin, Exp Neurol. 1997 October; 147(2):452-62 (botulinum toxin blockade caused epidermal thickness to be significantly reduced in the central area of the sole of the rat foot). Therefore, administration of botulinum toxin can effectively alter the thickness and thus the permeability of the skin.

In some embodiments, the methods of the present invention comprise the steps of calculating a dose of neurotoxin sufficient to increase the permeability of area of treatment (“enhancement dose”), and locally administering a neurotoxin to the area of treatment. Although examples of routes of administration and dosages are provided, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14^{th} edition, published by McGraw Hill). For example, the route and dosage for administration of a neurotoxin according to the present disclosed invention can be selected based upon criteria such as the solubility characteristics of the neurotoxin chosen as well as the area to be treated.

The present invention also includes the use of (a) Clostridial neurotoxins obtained or processed by bacterial culturing, toxin extraction, concentration, preservation, freeze drying, and/or reconstitution; and/or (b) modified or recombinant neurotoxins, that is neurotoxins that have had one or more amino acids or amino acid sequences deliberately deleted, modified or replaced by known chemical/biochemical amino acid modification procedures or by use of known host cell/recombinant vector recombinant technologies, as well as derivatives or fragments of neurotoxins so made. These neurotoxin variants retain the ability to inhibit neurotransmission between or among neurons, and some of these variants may provide increased durations of inhibitory effects as compared to native neurotoxins, or may provide enhanced binding specificity to the neurons exposed to the neurotoxins. These neurotoxin variants may be selected by screening the variants using conventional assays to identify neurotoxins that have the desired physiological effects of inhibiting neurotransmission.

Botulinum toxins for use according to the present invention can be stored in lyophilized, vacuum dried form in containers under vacuum pressure or as stable liquids. Prior to lyophilization the botulinum toxin can be combined with pharmaceutically acceptable excipients, stabilizers and/or carriers, such as albumin. The lyophilized material can be reconstituted with saline or water to create a solution or composition containing the botulinum toxin to be administered to the patient.

Although the composition may only contain a single type of neurotoxin, such as botulinum toxin type A, as the active ingredient to suppress neurotransmission, other therapeutic compositions may include two or more types of neurotoxins, which may provide improved enhancement (permeability factor) and subsequently an improved outcome of the treatment.

For example, a composition administered to a patient may include botulinum toxin type A and botulinum toxin type B. Administering a single composition containing two different neurotoxins may permit the effective concentration of each of the neurotoxins to be lower than if a single neurotoxin is administered to the patient while still achieving the desired therapeutic effects. The composition administered to the patient may also contain other pharmaceutically active ingredients, such as, protein receptor or ion channel modulators, in combination with the neurotoxin or neurotoxins.

These modulators may contribute to the reduction in neurotransmission between the various neurons. The compositions may also include agents that affect ion flux through voltage gated calcium channels, potassium channels, and/or sodium channels. Thus, the compositions can include one or more neurotoxins, such as botulinum toxins, in addition to ion channel receptor modulators that may reduce neurotransmission.

The neurotoxin may be administered by any suitable method as determined by the attending physician. The methods of administration permit the neurotoxin to be administered locally to a selected target tissue. Methods of administration include injection of a solution or composition containing the neurotoxin, as described above, and include implantation of a controlled release system that controllably releases the neurotoxin to the target tissue. Such controlled release systems reduce the need for repeat injections. Diffusion of biological activity of a botulinum toxin within a tissue appears to be a function of dose and can be graduated. Jankovic J., et al Therapy With Botulinum Toxin, Marcel Dekker, Inc., (1994), page 150. Thus, diffusion of botulinum toxin can be controlled to reduce potentially undesirable side effects that may affect the patient's cognitive abilities. For example, the neurotoxin can be administered so that the neurotoxin primarily effects neural systems believed to be involved in the generation of pain and/or inflammation, and does not have negatively adverse effects on other neural systems.

A polyanhydride polymer, GLIADEL® (Stolle R & D, Inc., Cincinnati, Ohio) a copolymer of poly-carboxyphenoxypropane and sebacic acid in a ratio of 20:80 has been used to make implants, and has been intracranially implanted to treat malignant gliomas. Polymer and BCNU can be co-dissolved in methylene chloride and spray-dried into microspheres. The microspheres can then be pressed into discs 1.4 cm in diameter and 1.0 mm thick by compression molding, packaged in aluminum foil pouches under nitrogen atmosphere and sterilized by 2.2 megaRads of gamma irradiation. The polymer permits release of carmustine over a 2-3 week period, although it can take more than a year for the polymer to be largely degraded. Brem, H., et al, Placebo-Controlled Trial of Safety and Efficacy of Intraoperative Controlled Delivery by Biodegradable Polymers of Chemotherapy for Recurrent Gliomas, Lancet 345; 1008-1012:1995.

Implants useful in practicing the methods disclosed herein may be prepared by mixing a desired amount of a stabilized neurotoxin (such as non-reconstituted BOTOX®) into a solution of a suitable polymer dissolved in methylene chloride. The solution may be prepared at room temperature. The solution can then be transferred to a Petri dish and the methylene chloride evaporated in a vacuum desiccators. Depending upon the implant size desired and hence the amount of incorporated neurotoxin, a suitable amount of the dried neurotoxin incorporating implant is compressed at about 8000 p.s.i. for 5 seconds or at 3000 p.s.i. for 17 seconds in a mold to form implant discs encapsulating the neurotoxin. See e.g. Fung L. K. et al., Pharmacokinetics of Interstitial Delivery of Carmustine 4-Hydroperoxycyclophosphamide and Paclitaxel From a Biodegradable Polymer Implant in the Monkey Brain, Cancer Research 58; 672-684:1998.

Local administration of a Clostridial toxin, such as a botulinum toxin, can provide a high, local therapeutic level of the toxin. A controlled release polymer capable of long term, local delivery of a Clostridial toxin to a target muscle permits effective dosing of a target tissue. A suitable implant, as set forth in U.S. Pat. No. 6,306,423 entitled “Neurotoxin Implant”, allows the direct introduction of a chemotherapeutic agent to a target tissue via a controlled release polymer. The implant polymers used are preferably hydrophobic so as to protect the polymer incorporated neurotoxin from water induced decomposition until the toxin is released into the target tissue environment.

The amount of a neurotoxin selected for local administration to a target tissue according to the present disclosed invention can be varied based upon criteria such as the location of the treatment, or the solubility characteristics of the agent or formulation chosen, as well as the age, sex, weight and health of the patient. For example, the extent of the area of muscle tissue influenced is believed to be proportional to the volume of neurotoxin injected, while the quantity of the relaxation effect is, for most dose ranges, believed to be proportional to the concentration of a Clostridial toxin administered. In this invention, the dose of botulinum toxin type A is determined by measuring the skin surface (cm²) and multiplying the total area by, for example, 1 unit of BOTOX. The drug is administered so that the amount per injection site equals the diameter of diffusion of the drug (for botulinum toxin type A 1 U=1 cm²).

Methods for determining the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14^{th} edition, published by McGraw Hill).

According to our invention, the neurotoxin (such as a botulinum toxin serotype A, B, C₁, D, E, F or G) can be injected locally (e.g., intramuscular injection) into or in the vicinity of the intended treatment.

In some embodiments, the neurotoxin can be administered intradermally and/or subdermally. Further, the neurotoxin can be administered at one or multiple sites.

In some embodiments, the methods of the present invention improves the outcome of a treatment as compared to a treatment that is not accompanied by an enhancement step with a botulinum toxin. An improvement of the treatment outcome is by more than about 5%, preferably more than about 20%, even more preferably more than 50%, as compared to using the same treatment but without a neurotoxin step. For example, patients being treated with methods of the present invention would experience improvements in the reduction of both fine and deep glabellar folds following collagen injections by using an enhancement step (e.g. botulinum toxin) versus just the collagen injection alone.

In some embodiments, the enhancement effects provided by the neurotoxin can persist for a relatively long period of time, for example, for more than two months, and potentially for several years. For example, patients being treated with methods of the present invention would experience improvements in the duration of the treatment outcome wherein, future treatments would be for example 1 year later versus 6 months.

In some embodiments, the enhancement effects provided by the neurotoxin allow a decrease in exposure to a treatment, for example, laser hair removal. Patients being treated with methods of the present invention would experience more rapid rates of hair removal thus obviating the need for multiple visits, as well as a reduction of potential adverse events (e.g., redness, pain, inflammation), versus the treatment alone.

Dermabrasion and dermaplaning help to “refinish” the skin's top layers through a method of controlled surgical scraping. The treatments soften the sharp edges of surface irregularities, giving the skin a smoother appearance. Dermabrasion and dermaplaning can smooth scars left by acne, accidents, or previous surgery, as well as fine facial wrinkles, especially those around the mouth. It's also sometimes used to remove the pre-cancerous growths called keratoses. Dermaplaning is commonly used to treat deep acne scars.

Both dermabrasion and dermaplaning can be performed on small areas of skin or on the entire face. They can be used alone, or in conjunction with other procedures such as facelift, scar removal or revision, or chemical peel. Dermabrasion and dermaplaning are normally safe. The most common risk is a change in skin pigmentation. Permanent darkening of the skin, usually caused by exposure to the sun in the days or months following surgery, may occur in some patients. Improvements in dermabrasion and dermaplaning by administration of a neurotoxin, could reduce the amount of exposure time of the treatment, thus improving the outcome of the treatment.

For example, clinical trials show that a non-animal stabilized hyaluronic acid (NASHA) is effective for up to one year after the treatment of wrinkles. However, the duration of treatment often depends on many factors, such as the structure and permeability of the skin, lifestyle and age, degree of perfection demanded by the patient and the injection technique. Follow-up treatment is required after 6-12 months. Enhancement of the skin area with a neurotoxin, by means of the methods disclosed herein, would enhance the effectiveness of the collagen treatment, e.g., greater reduction of skin folds.

In some embodiments, the methods of the present invention can enhance the therapeutic effects of a procedure or agent by enhancing the treatment area with a neurotoxin. The methods comprise the steps of locally administering a neurotoxin to the patient (“enhancement step”) by use of a novel formula disclosed herein. The methods further comprise the steps of using a novel rating scale disclosed herein, to determine the amount of enhancement and amount of adjustment to the subsequent treatment or agent.

In some embodiments the enhancement may be during to the step of administering the agent or procedure. In some embodiments, the enhancement of the area may be prior to the step of administering a procedure or treatment. In some embodiments, the enhancement via administration of a neurotoxin may be after the step of administering the agent or procedure.

In some embodiments, the amount of time required to achieve the current dose is provided by calculating the units necessary to sufficiently enhance the area of treatment.

In some embodiments, the treatment is an application of one or more of the following non-limiting, exemplary agent (and/or formulation thereof):

1. corticosteroids emollient

2. botanical agent

3. wrinkle modifier

4. growth factor

5. moisturizer

6. peptide

7. antioxidant

8. keratolytic

9. retinoid

10. deoxyribonucleic acid

11. acne agent

12. enzyme

13. ascorbic acid

14. anticellulite

15. growth factor

16. glycosaminoglycan

17. facial emollient

In some embodiments, the treatment is an administration of one or more dermatologic or cosmetic treatments including but not limited to;

1. Facial rejuvenation procedures

2. Thermage treatment

3. filler-based treatment and procedures

4. high intensity focused ultrasound treatment

5. hormonal treatment for facial enhancement

6. laser treatment for facial rejuvenation procedures

7. anesthetic treatment prior to facial enhancement therapies

8. DNA facial enhancement treatment

9. intense pulsed light photo rejuvenation procedures

10. microabrasion and dermabrasion therapies

11. laser treatment in hair removal procedures

12. tattoo removal treatments

13. hair removal treatment

14. hair loss treatment

15. anticellulite treatment

16. varicose vein treatment

17. aesthetician treatments

18. massage treatment
physical and occupational therapy

19. chiropractic procedures

In addition, this invention may be applied to the following examples:

1. Non-cutaneous and cutaneous cell proliferative disorders

2. Corticosteroid treatment for psoriasis

3. Retinoid treatment for psoriasis

With respect to applying the present methods for dermatologic or cosmetic procedures or treatments in general, our invention is preferably practiced by administering a botulinum toxin directly to a location where the intending treatment is to be performed. Without wishing to be bound by theory a physiological mechanism can be proposed for the efficacy of the present invention. Peripheral administration of botulinum toxin can effectively relax (atrophy); muscle spindle fibers; dermal and hypodermal layers; skeletal muscle fibers; smooth muscles surrounding glands and hair follicles, thus enlarging the spaces between the dermis, muscle layers, hair shafts, and glands (e.g., increase permeability). The mechanism by which botulinum toxin effectively alters the permeability rates is by inhibition of the release of neurotransmitters such as acetycholine and perhaps other mediators found in the epidermal layers including; substance P, cGRP and VIP. As a result of this inhibition, botulinum toxin effectively “relaxes” or enhances the intended area of treatment and allows greater permeation of the treatment and subsequently resulting in a similar or better treatment effect.

Several physiological responses can facilitate drug penetration through the skin. These include: an increase in skin permeability; increases in body fluid circulation; dilation and increased permeability through blood vessel walls. These responses can result in an improvement in the solubility of most drugs; and an increase in the release rate of the drug from local skin tissue into systemic circulation. There are a number of agents that can be locally applied to alter the permeability of the skin and allow for greater penetration of a therapeutic compound.

It is known that muscles have a complex system of innervation and sensory output. Thus, anterior motor neurons located in each segment of the anterior horns of the spinal cord gray matter give rise to efferent alpha motor neurons and efferent gamma motor neurons that leave the spinal cord by way of the anterior roots to innervate skeletal (extrafusal) muscle fibers. The alpha motor neurons cause contraction of extrafusal skeletal muscle fibers while the gamma motor neurons innervate the intrafusal fibers of skeletal muscle. As well as excitation by these two type of efferent anterior motor neuron projections, there are additional, afferent sensory neurons which project from muscle spindle and golgi tendon organs and act to transmit information regarding various muscle parameter status to the spinal cord, cerebellum and cerebral cortex. These afferent motor neurons which relay sensory information from the muscle spindle include type Ia and type II sensory afferent neurons. See e.g. pages 686-688 of Guyton A. C. et al., Textbook of Medical Physiology, W.B. Saunders Company 1996, ninth edition.

Significantly, it has been determined that a neurotoxin, i.e., a botulinum toxin, can act to reduce transmission of sensory information from muscle type Ia afferent neurons. Aoki, K., Physiology and pharmacology of therapeutic botulinum neurotoxins, in Kreyden, O., editor, Hyperhidrosis and botulinum toxin in dermatology, Basel, Karger; 2002; 30: pages 107-116, at 109-110. And it has been hypothesized that botulinum toxin can have a direct effect upon muscle cell sensory afferents and modify signals from these afferents to the central nervous system. See e.g. Brin, M., et al., Botulinum toxin type A: pharmacology, in Mayer N., editor, Spasticity: etiology, evaluation, management and the role of botulinum toxin, 2002; pages 110-124, at 112-113; Cui, M., et al., Mechanisms of the antinociceptive effect of subcutaneous BOTOX®: inhibition of peripheral and central nociceptive processing, Naunyn Schmiedebergs Arch Pharmacol 2002; 365 (supp 2): R17; Aoki, K., et al., Botulinum toxin type A and other botulinum toxin serotypes: a comparative review of biochemical and pharmacological actions, Eur J. Neurol 2001: (suppl 5); 21-29. Thus, it has been demonstrated that botulinum toxin can cause an altered sensory output from muscle to CNS and brain. Importantly, the sensory neurons from which afferent output is to be inhibited by a method according to the present invention need not be located on or within a muscle, but can be in an intradermal or subdermal location.

It is our hypothesis that, a local administration of a neurotoxin, e.g., a botulinum toxin, to muscle spindle fibers, dermal and hypodermal layers of the skin, effectively relaxes muscle fibers, and smooth muscles surrounding glands and hair follicles, thus effectively increasing the permeability of the surrounding area.

Significantly, a method within the scope of the present invention can provide improved treatment results. Improved treatment results can be defined as an improvement measured by factors such as a decrease in the amount of time for the treatment application, reduction in the amount of agent or agents used in the treatment, and generally an improved outcome.

According to our invention, the methods comprise the steps of calculating the area of treatment and amount of neurotoxin units necessary to increase permeability. To guide the practitioner, typically, no less than 1 U to 5 U of botulinum toxin type A is administered per 1 cm² area. In some embodiments, the amount neurotoxin necessary to administered is calculated as no less than 10 U of botulinum toxin type B per 1 cm² area.

According to our invention the extent of the area influenced is proportional to the volume of neurotoxin injected, while the amount of enhancement is, for most dose ranges, believed to be proportional to the dose of botulinum toxin which is determined by measuring the skin surface (cm²) area of the region where the treatment is to be applied, thus “enhanced”, and multiplying the total area (cm²) by the neurotoxin units specified as disclosed above. This formula calculates the amount of neurotoxin per injection site equals the diffusion radius of the drug; for example; botulinum toxin type A 1 U to 5 U=1 cm².

In some embodiments, the toxin administered is a dose between for example, 20-75 U of botulinum toxin type B, where the determined amount per injection site equals the diffusion radius of the drug.

In some embodiments, the toxin administered is a dose between 1-5 U of a 150 kD botulinum toxin type A neurotoxin only formulation where the determined amount per injection site equals the diffusion radius of the drug.

In some embodiments, the methods of the present invention improves the outcome of the treatment by more than about 10% to about 95% as compared to the outcome of the treatment not accompanied by a neurotoxin enhancement step. For example, patients being treated with methods of the present invention would experience a greater and more rapid reduction in glabellar folds following application of a treatment consisting of a wrinkle modifier. In some embodiments, the therapeutic enhancement effects provided by the neurotoxin can persist for a relatively long period of time, for example, for more than two months, and potentially for several years.

The amount of the neurotoxin administered according to a method within the scope of the disclosed invention can vary according to the particular area being treated, including location and type of treatment and other various patient variables including size, weight, age, and responsiveness to therapy. To guide the practitioner, typically, no less than about 1 unit per square cm of area and no more than about 25 units of a botulinum toxin type A (such as BOTOX® or Xeomin®) is administered per injection site (i.e. to each muscle portion injected), per patent treatment session. For a botulinum toxin type A such as DYSPORT®, no less than about 2 units and no more about 125 units of the botulinum toxin type A are administered per injection site, per patent treatment session. For a botulinum toxin type B such as MYOBLOC®, no less than about 40 units and no more about 1500 units of the botulinum toxin type B are administered per injection site, per patent treatment session. Less than about 1, 1, 2 or 40 units (of BOTOX® or Xeomin®, DYSPORT® and MYOBLOC® respectively) can fail to achieve a desired therapeutic effect, while more than about 25, 25, 125 or 1500 units (of BOTOX®, or Xeomin®, DYSPORT® and MYOBLOC® respectively) can result in significant muscle hypotonicity, weakness and/or paralysis.

More preferably: for BOTOX® or Xeomin®, no less than about 2 units and no more about 20 units of a botulinum toxin type A; for DYSPORT® no less than about 4 units and no more than about 100 units, and; for MYOBLOC®, no less than about 80 units and no more than about 1000 units are, respectively, administered per injection site, per patent treatment session.

Even more preferably: for BOTOX® or Xeomin®, no less than about 5 units and no more about 15 units of a botulinum toxin type A; for DYSPORT® no less than about 20 units and no more than about 75 units, and; for MYOBLOC®, no less than about 200 units and no more than about 750 units are, respectively, administered per injection site, per patent treatment session. It is important to note that there can be multiple injection sites (i.e. a pattern of injections) for each patient treatment session.

Generally, the total amount of BOTOX®, Xeomin®, DYSPORT® or MYOBLOC®, suitable for administration to a patient according to the methods of the invention disclosed herein should not exceed about 300 units, about 1,500 units or about 15,000 units respectively, per treatment session.

In some embodiments, the “enhancement step” is calculated to occur prior to the treatment; within about an amount of time equal to that required to achieve the optimal effect of the neurotoxin, wherein the treatment would then follow.

In some embodiments, the amount of time required to achieve the desired effect is provided by calculating the maximal effect of the neurotoxin dose; a range of about 1 day to 14 days.

In some embodiments, the “enhancement step” occurs prior to the step of administering the treatment. For example, no less than 1 U of botulinum toxin type A per 1 square centimeter area of the intended area of treatment, is applied, followed by the treatment.

In some embodiments, the “enhancement step” occurs during to the step of administering the treatment. For example, no less than 1 U of botulinum toxin type A per 1 square centimeter area of the intended area of treatment, is applied, during the treatment.

In some embodiments, the “enhancement step” occurs after the step of administering the agent or procedure. For example, no less than 1 U of botulinum toxin type A per 1 square centimeter area of the intended area of treatment, is applied, after the treatment

In some embodiments, the “enhancement step” is calculated to occur prior to the treatment; within about an amount of time equal to that required to achieve the optimal effect of the neurotoxin, wherein the treatment would then follow, followed by another “enhancement step”.

For example, no less than 1 U of botulinum toxin type A per 1 square centimeter area is applied to the intended area of treatment; the treatment or intended procedure is applied; one or more “enhancement steps” would then follow.

In some embodiments, “enhancement step” occurs within about an amount of time equal to about 2 hours to 4 weeks prior to the procedure or treatment.

In some embodiments, “enhancement step” within about an amount of time equal to about 2 hours to 4 weeks prior to the procedure or treatment, followed by the treatment, with one or more “enhancement steps” after the treatment.

In some embodiments, “enhancement step” occurs at about the same time as the procedure or treatment, followed by one or more “enhancement steps” after the procedure or treatment.

In some embodiments, “enhancement step” occurs at about the same time as the procedure or treatment, followed by one or more “enhancement steps” without repeat of the treatment.

In some embodiments, “enhancement step” occurs after the procedure or treatment, followed by one or more “enhancement steps” with or without repeat of the treatment.

In some embodiments, the, “enhancement step” may occur as a stepwise process. For example, there are situations where a particular desired effect from a procedure or treatment is not immediately achievable because the patient is not able to tolerate the procedure or treatment. An application of a stepwise “enhancement step” may be effective in a situation like this. A stepwise “enhancement step” comprises an administration of a neurotoxin in alternation with the treatment for as many times as needed. For example, a stepwise “enhancement step” in accordance with the present invention comprises the following steps in order:

### (1) Administering a neurotoxin

### (2) Application of the treatment; e.g. laser therapy

### (3) Administering a neurotoxin

### (4) Application of the procedure or treatment, etc.

In some embodiments, the “enhancement step” would precede application of one or more treatments (e.g., dermaplaning) followed by one or more formulation or agents (e.g., collagen filler).

In some embodiments, the “enhancement step” would precede application of one or more formulations or agents (e.g., retinoid formulation) followed by one or more treatments (e.g., laser therapy).

In some embodiments, where multiple procedures are done, the “enhancement step” would precede application of one of the procedures (e.g., laser therapy and thermage treatment).

In some embodiments, where multiple procedures are done, the “enhancement step” would precede application of more than one or all of the procedures.

In some embodiments, where multiple formulations or agents are applied, the “enhancement step” would precede application of one of the formulations or agents

In some embodiments, where multiple formulations or agents are applied, the “enhancement step” would precede application of more than one or all of the formulations or agents.

In some embodiments, where multiple procedures are done, the “enhancement step” would precede application of more than one or all of the procedures.

The methods of the present invention are effective in enhancing the desired effect of a dermatologic or cosmetic treatment. Further, the methods of the present invention are effective in enhancing the desired effect of a dermatologic or cosmetic formulation or agent.

An example of multiple treatments includes for example; application of a botulinum toxin for enhancement of the receptive area, followed by a corticosteroid, then by an emollient viscoelastic composition; applied in a single or in multiple applications of the composition to the facial region to treat, in particular, marrionette lines, glabellar lines, crows feet, and for brow lift.

Optionally, according to our invention, following the appropriate amount of botulinum toxin injection, the amount of enhancement is rated (quantified) using a 3 point “enhancement rating scale”. In some embodiments, the present inventions relates to the use of a rating scale to determine the amount of enhancement achieved. In some embodiments, the present inventions relates to the use of a rating scale to determine the reduction of the usual amount of an agent or time of exposure of a treatment, and adjustments therein. In some embodiments, the methods to determine the amount of enhancement obtained is by use of an optional device to measure tension of the skin, the tension is determined and compared to the value prior to botulinum toxin injection. If the amount of tension is reduced between, for example, 0 to 30 percent, the enhancement score would equal one. If the amount of tension is reduced between 31 to 60 percent, the enhancement score would equal two. If the amount of tension is reduced 60 percent or more the enhancement score would equal three.

In some embodiments, the methods of the present invention, would utilize a method of determining tension by use of finger touch and rating according to a 3 point “enhancement rating scale”.

In some embodiments, the methods to determine the amount of enhancement is utilized to determine the amount that a procedure can be reduced, e.g., decrease time (exposure) of a laser therapy application.

According to our invention, the adjustment to a treatment is proportional to the enhancement score, e.g., a score of one equals a reduction of 0 to 30 percent; a score of 2 equals an adjustment of 30-60 percent, and a score of 3 equals an adjustment of 60 to 100 percent.

In some embodiments, the adjustment to a treatment is proportional to the enhancement score, e.g., a score of one equals a reduction of 0 to 15 percent; a score of 2 equals an adjustment of 15-30 percent, and a score of 3 equals an adjustment of 30 to 100 percent.

In some embodiments, the adjustment to a treatment is proportional to the enhancement score, e.g., a score of one equals a reduction of 0 to 25 percent; a score of 2 equals an adjustment of 25-50 percent, and a score of 3 equals an adjustment of 50 to 100 percent.

In some embodiments, the adjustment to a treatment is proportional to the enhancement score, e.g., a score of one equals a reduction of 0 to 40 percent; a score of 2 equals an adjustment of 40-80 percent, and a score of 3 equals an adjustment of 80 to 100 percent.

After the administration of the neurotoxin, and the procedure or treatment, the methods comprise an optional step of enhancing further with a subsequent amount of neurotoxin.

### EXAMPLES

The following non-limiting example provides those of ordinary skill in the art with specific preferred methods to treat conditions within the scope of the present invention and are not intended to limit the scope of the invention. In the following examples various modes of non-systemic administration of a neurotoxin can be carried out. For example, by intramuscular injection, subcutaneous injection or by implantation of a controlled release implant.

### Example 1

### Improved Outcomes of Dermabrasion Therapy by Enhancement of the Receptive Treatment Area

A 36 year old female patient who has a history of cystic acne during adolescence was seen in the cosmetic clinic. Upon examination, the clinician noted that the skin in the facial area and forehead region had numerous scars; some pitted and others had a dark discoloration. It was also noted that there was the appearance of rosacea and therefore a concern on whether dermabrasion therapy might further scar or disrupt the skin area, thus the clinician planned to do an enhancement step prior to the procedure to avoid these potential outcomes. The clinician calculated the surface area to be 100 cm² and therefore further multiplied this total by 2.0 U of a botulinum toxin type A; a total dose of 100 U. The botulinum toxin was distributed evenly over the forehead region, with two injections at the superior border of the glabella area. In addition, 4 injections were spaced evenly on both the left and right regions of the face. The patient returned 14 days later and the clinician estimated an enhancement score of 3 by way of a finger tension test. Therefore, a 70 percent reduction in the amount of exposure time was determined and the treatment was administered. The patient reported minimal pain and very little redness afterwards. On follow up one month later, the areas of treatment were clear and only minimal scarring was evident.

### Example 2

### Laser Treatment of Fine Wrinkles Using an Enhancement of the Receptive Area

A 45 year old female patient with damage to the skin in the facial area secondary to significant sun exposure has fine wrinkles around the eyes and mouth desired laser treatment at a cosmetic procedures clinic. There is a concern about her history of skin sensitivity and whether the laser treatment might cause redness, swelling and possibly scarring.

To optimize treatment and to limit the duration of exposure to the carbon dioxide (CO₂) laser device, the clinician employs a method to enhance the receptive area of treatment. The dose of botulinum toxin type A is determined by measuring the skin surface (50 cm²) and multiplied the total by 1 unit of BOTOX (total 50 U). The drug is administered as 25 injections so that the amount per injection site equaled the diameter of diffusion of the drug (1 U=2 cm²). The patient returns 10 days later for her laser treatment. Upon inspection, the clinician determines an enhancement score or 2, and calculated a 40% reduction in usual exposure time (30 minutes) of the laser treatment. Following application of local anesthesia, the clinician applies the laser treatment for only 12 minutes. Upon follow up 30 days later, the patient reports no discomfort, redness or scarring. Upon inspection the previously visible fine lines around both the eyes and mouth are not noticeable.

### Example 3

### Enhancement of a Deep and Fine Line Wrinkle Treatment

A 45 year old male patient, who has been a smoker for 15 years and a landscaper in southern United States had significant skin folds in the forehead and glabellar region. He has a recent career change and is going to be an actor and desires to improve his appearance. Since the patient would be leaving for a rural area, he would not be able to return for follow up treatments. Therefore to optimize the treatment, the clinician calculates the area of treatment to be 50 cm². The clinician calculates 100 U (2 U/cm²) of botulinum toxin type A, and administers the toxin at 15 sites evenly over the area. After a 30 minute period during which the blebs from the injections subsides, the clinician injects a standard amount of a collagen filler preparation, altering the site of injection of the previous botulinum toxin. 2 months later upon phone follow-up, the patient reports that all of the visible fine lines has diminished and nearly all of his deep lines has disappeared.

Although the present invention has been described in detail with regard to certain preferred methods, other embodiments, versions, and modifications within the scope of the present invention are possible. For example, a wide variety of neurotoxins can be effectively used in the methods of the present invention. Additionally, the present invention includes local administration methods to enhance the receptive field area (enhance permeability or penetration) wherein two or more neurotoxins, such as two or more botulinum toxins, are administered concurrently or consecutively. For example, botulinum toxin type A can be administered until a loss of clinical response or neutralizing antibodies develop, followed by administration of botulinum toxin type B. Alternately, a combination of any two or more of the botulinum serotypes A-G can be locally administered to control the onset and duration of the desired therapeutic result. Furthermore, non-neurotoxin compounds can be administered prior to, concurrently with or subsequent to administration of the neurotoxin to proved adjunct effect such as enhanced or a more rapid onset of denervation before the neurotoxin, such as a botulinum toxin, begins to exert its therapeutic effect.

A method for treating skin according to the invention disclosed herein has many benefits and advantages, including the following:

1. Effects of several different cosmetic or dermatologic treatments can be improved whereby the amounts and or exposure times to such treatments may be reduced significantly.

2. Amounts of dermatologic or cosmetic formulations or agents can be dramatically reduced or eliminated, thereby reducing any potential adverse events.

3. Effects of various dermatologic or cosmetic treatments or procedures including various formulations and agents may be significantly improved for at least a period of about two to about six months per injection of neurotoxin and from about one year to about five years upon use of a controlled release neurotoxin implant.

Our invention also includes within its scope the use of a neurotoxin, such as a botulinum neurotoxin, to enhance the effects of a concomitant or subsequent agent, formulation or cosmetic procedure by local administration of the botulinum toxin.

All references, articles, patents, applications and publications set forth above are incorporated herein by reference in their entireties.

Accordingly, the spirit and scope of the following claims should not be limited to the descriptions of the preferred embodiments set forth above.

What is claimed is:

1. A method for enhancing a skin treatment for a human patient in need thereof, the method comprising the step of administering a neurotoxin to a skin area designated for skin treatment prior to the skin treatment, wherein a dose of neurotoxin administered is calculated by
(a) determining a surface area (centimeter square) of the designated skin area, and
(b) multiplying the surface area by a unit of neurotoxin that is equivalent to about 1-5 units of botulinum toxin type A,
thereby prolonging a treatment result of the skin treatment at the designated skin area.

2. The method of claim 1 wherein the skin treatment comprises administering a dermatologic agent.

3. The method of claim 2 wherein the skin treatment comprises administering a skin filler.

4. The method of claim 2 wherein the dermatologic agent comprises a corticosteroids emollient, wrinkle modifiers, growth factor, moisturizer, peptide, antioxidant, keratolytic agent, retinoid, deoxyribonucleic acid, acne agents, enzymes, ascorbic acid, anticellulite, glycosaminoglycans, hyaluronic acid, crossed linked hyaluronic acid or combination thereof.

5. The method of claim 1 wherein the skin treatment comprises applying a dermatologic procedure.

6. The method of claim 5 wherein the dermatologic procedure comprises a varicose vein treatment, thermage treatment in facial rejuvenation procedures, filler-based treatment, high intensity focused ultrasound treatment, hormonal treatment for facial enhancement, laser treatment for hair removal, laser treatment for facial rejuvenation, tattoo removal treatments, hair removal treatment, microabrasion and dermabrasion therapies, hair loss treatment, DNA facial enhancement treatment, intense pulsed light photo rejuvenation procedures, anesthetic treatment prior to facial enhancement therapies, anticellulite treatment, facial enhancement, aesthetician treatments, massage treatment, physical and occupational therapy, chiropractic procedures, or combination thereof.

7. The method of claim 1 wherein the neurotoxin is a botulinum toxin type A, B, C₁, D, E, F or G.

8. The method of claim 1 wherein the neurotoxin is a botulinum toxin type A.

9. The method of claim 1 wherein the neurotoxin is administered intramuscularly, transdermally or subcutaneously.

10. A method for enhancing a skin treatment for a human patient in need thereof, the method comprising the step of administering a neurotoxin toxin to a skin area designated for skin treatment, thereby prolonging the treatment result of the skin treatment at the designated skin area,
wherein the skin treatment comprises administering a dermatologic agent comprising a corticosteroids emollient, wrinkle modifiers, growth factor, moisturizer, peptide, antioxidant, keratolytic agent, retinoid, deoxyribonucleic acid, acne agents, enzymes, ascorbic acid, anticellulite, or combination thereof.

11. The method of claim 10 wherein the neurotoxin is a botulinum toxin type A, B, C₁, D, E, F or G.

12. The method of claim 10 wherein the neurotoxin is a botulinum toxin type A.

13. The method of claim 10 wherein the neurotoxin is administered intramuscularly, transdermally or subcutaneously.

14. A method for enhancing a skin treatment for a human patient in need thereof, the method comprising the step of administering a botulinum toxin to a skin area designated for skin treatment, thereby prolonging the treatment result of the skin treatment at the designated skin area,
wherein the skin treatment comprises a varicose vein treatment, thermage treatment in facial rejuvenation procedures, filler-based treatment, high intensity focused ultrasound treatment, hormonal treatment for facial enhancement, laser treatment for hair removal, laser treatment for facial rejuvenation, tattoo removal treatments, hair removal treatment, microabrasion and dermabrasion therapies, hair loss treatment, DNA facial enhancement treatment, intense pulsed light photo rejuvenation procedures, anesthetic treatment prior to facial enhancement therapies, anticellulite treatment, facial enhancement, aesthetician treatments, massage treatment, physical and occupational therapy, chiropractic procedures, or combination thereof.

15. The method of claim 14 wherein the neurotoxin is a botulinum toxin type A, B, C₁, D, E, F or G.

16. The method of claim 14 wherein the neurotoxin is a botulinum toxin type A.

17. The method of claim 14 wherein the neurotoxin is administered intramuscularly, transdermally or subcutaneously.

18. A method for reducing an amount of a dermatologic agent used during treatment of a skin condition in a human patient in need thereof, the method comprising the step of administering a neurotoxin to a skin area designated for skin treatment,
thereby reducing the amount of dermatologic agent used to obtain a treatment result at the designated skin area, wherein a higher amount of an identical dermatologic agent is required to obtain the same treatment result if the neurotoxin is not administered to the designated skin area.

19. The method of claim 18 wherein the dermatologic agent comprises a corticosteroids emollient, wrinkle modifiers, growth factor, moisturizer, peptide, antioxidant, keratolytic agent, retinoid, deoxyribonucleic acid, acne agents, enzymes, ascorbic acid, anticellulite, glycosaminoglycans, hyaluronic acid, crossed linked hyaluronic acid or combination thereof.

20. The method of claim 18 wherein the neurotoxin is a botulinum toxin type A, B, C₁, D, E, F or G.

21. The method of claim 18 wherein the neurotoxin is a botulinum toxin type A.

22. The method of claim 18 wherein the neurotoxin is administered intramuscularly, transdermally or subcutaneously.

23. A method for reducing an amount of exposure time to a dermatologic procedure during treatment of a skin condition in a human patient in need thereof, the method comprising the step of administering a neurotoxin to a skin area designated for skin treatment,
thereby reducing the amount of exposure time to the dermatologic procedure to obtain a treatment result at the designated skin area,
wherein a longer amount of exposure time to an identical dermatologic procedure is required to obtain the same treatment result if the neurotoxin is not administered to the designated skin area.

24. The method of claim 23 wherein the dermatologic procedure comprises a varicose vein treatment, thermage treatment in facial rejuvenation procedures, filler-based treatment, high intensity focused ultrasound treatment, hormonal treatment for facial enhancement, laser treatment for hair removal, laser treatment for facial rejuvenation, tattoo removal treatments, hair removal treatment, microabrasion and dermabrasion therapies, hair loss treatment, DNA facial enhancement treatment, intense pulsed light photo rejuvenation procedures, anesthetic treatment prior to facial enhancement therapies, anticellulite treatment, facial enhancement, aesthetician treatments, massage treatment, physical and occupational therapy, chiropractic procedures, or combination thereof.

25. The method of claim 23 wherein the neurotoxin is a botulinum toxin type A, B, C₁, D, E, F or G.

26. The method of claim 23 wherein the neurotoxin is a botulinum toxin type A.

27. The method of claim 23 wherein the neurotoxin is administered intramuscularly, transdermally or subcutaneously.

28. A method for treating a skin condition for a human patient in need thereof, the method comprising the steps of
(a) administering a neurotoxin to a skin area designated for skin treatment,
(b) determining a muscle tone at the designated skin area,
(c) subjecting the designated skin area to a dermatologic agent or procedure.

29. The method of claim 28 wherein the skin treatment comprises administering a dermatologic agent.

30. The method of claim 28 wherein the skin treatment comprises administering a skin filler.

31. The method of claim 29 wherein the dermatologic agent comprises a corticosteroids emollient, wrinkle modifiers, growth factor, moisturizer, peptide, antioxidant, keratolytic agent, retinoid, deoxyribonucleic acid, acne agents, enzymes, ascorbic acid, anticellulite, glycosaminoglycans, hyaluronic acid, crossed linked hyaluronic acid or combination thereof.

32. The method of claim 28 wherein the skin treatment comprises applying a dermatologic procedure.

33. The method of claim 32 wherein the dermatologic procedure comprises a varicose vein treatment, thermage treatment in facial rejuvenation procedures, filler-based treatment, high intensity focused ultrasound treatment, hormonal treatment for facial enhancement, laser treatment for hair removal, laser treatment for facial rejuvenation, tattoo removal treatments, hair removal treatment, microabrasion and dermabrasion therapies, hair loss treatment, DNA facial enhancement treatment, intense pulsed light photo rejuvenation procedures, anesthetic treatment prior to facial enhancement therapies, anticellulite treatment, facial enhancement, aesthetician treatments, massage treatment, physical and occupational therapy, chiropractic procedures, or combination thereof.

34. The method of claim 28 wherein an amount of neurotoxin administered is inversely proportional to a degree of muscle tone.

35. The method of claim 28 wherein the neurotoxin is a botulinum toxin type A, B, C₁, D, E, F or G.

36. The method of claim 28 wherein the neurotoxin is a botulinum toxin type A.

37. The method of claim 28 wherein the neurotoxin is administered intramuscularly, transdermally or subcutaneously.
